(19) United States
(12) Patent Application Publication
(10) Pub. No.: US 2015/0056261 A1
(43) Pub. Date: Feb. 26, 2015

(54) SILK MEDICAL DEVICES

(71) Applicant: Allergan, Inc., Irvine, CA (US)

(72) Inventor: Monica Serban, Melrose, MA (US)

(73) Assignee: Allergan, Inc., Irvine, CA (US)

(21) Appl. No.: 13/973,818

(22) Filed: Aug. 22, 2013

(51) Int. Cl. A61L 27/22; B32B 38/18; B32B 37/24

(52) U.S. Cl. CPC A61L 27/227; B32B 37/24; B32B 38/18; A61L 2300/252; B32B 2305/182; B32B 2307/73; B32B 2317/10; B32B 2535/00
USPC 424/423; 156/148

(57) ABSTRACT

Laminate medical devices and methods using such devices to support soft tissues and/or to reduce formation of post-operative adhesions. The medical devices can comprise a layer of a knitted silk mesh to which has been fused a water soluble silk film.

### BACKGROUND

The present invention relates to implantable silk devices. In particular the present invention relates to multi-laminate silk devices comprising one or more of a silk film, a silk fiber or fabric and hyaluronic acid or other macromolecules (such as for example dextran, heparin and sulphates thereof) in various combinations, as well as methods for making and using, for example in abdominal surgery.

Silk is a natural (non-synthetic) protein made of high strength fibroin fibers with mechanical properties similar to or better than many of synthetic high performance fibers. Silk is stable at physiological temperatures in a wide range of pH, and is insoluble in most aqueous and organic solvents. As a protein, unlike the case with most if not all synthetic polymers, the degradation products (e.g. peptides, amino acids) of silk are biocompatible. Silk is non-mammalian derived and carries far less bioburden than other comparable natural biomaterials (e.g. bovine or porcine derived collagen). Silk, as the term is generally known in the art, means a filamentous fiber product secreted by an organism such as a silkworm or spider. Silks can be made by certain insects such as for example Bombyx mori silkworms, and Nephilia clavipes spiders. There are many variants of natural silk. Fibroin is produced and secreted by a silkworm's two silk glands. As fibroin leaves the glands it is coated with sericin a glue-like substance. Spider silk is produced as a single filament lacking the immunogenic protein sericin.

Silk has been used in biomedical applications. The Bombyx mori species of silkworm produces a silk fiber (a “bave”) and uses the fiber to build its cocoon. The bave as produced include two fibroin filaments or broins which are surrounded with a coating of the gummy, antigenic protein sericin. Silk fibers harvested for making textiles, sutures and clothing are not sericin extracted or are sericin depleted or only to a minor extent and typically the silk remains at least 10% to 26% by weight sericin. Retaining the sericin coating protects the frail fibroin filaments from fraying during textile manufacture. Hence textile grade silk is generally made of sericin coated silk fibroin fibers. Medical grade silkworm silk is used as either as virgin silk suture, where the sericin has not been removed, or as a silk suture from which the sericin has been removed and replaced with a wax or silicone coating to provide a barrier between the silk fibroin and the body tissue and cells.

Hyaluronic acid (HA) (synonymously hyaluron or hyaluronate) is a naturally occurring glucosaminoglycan that has been used as a constituent of a dermal filler for wrinkle reduction and tissue volumizing. Hyaluronan is an anionic, nonsulfated glycosaminoglycan distributed widely throughout connective, epithelial, and neural tissues. Polymeric hyaluronic acid can have a molecular weight of several million Daltons. An individual can typically have about 15 grams of hyaluronan in his body about a third of which every day is degraded by endogenous enzymes and free radicals within a few hours or days and replaced by hyaluronic acid newly synthesized by the body.

Bioconjugate Chemistry, 2010, 21, 240-247: Joem Y., et al., Effect of cross-linking reagents for hyaluronic acid hydrogel dermal fillers on tissue augmentation and regeneration, discusses use of a particular cross-linker HMDA to prepare a cross-linked hyaluronic acid dermal filler, and also discloses use of a variety of hyaluronic acid cross linkers and hyaluronic activators including BDDE and EDC.

Carbohydrate Polymers, 2007, 70, 251-257: Jeon, O., et al., Mechanical properties and degradation behaviors of hyaluronic acid hydrogels cross-linked at various cross-linking densities, discusses properties of hyaluronic acid cross linked with a polyethylene glycol diamine (a PEG-diamine).

J. Am. Chem. Soc., 1955, 77 (14), 3908-3913: Schroeder W., et al., The amino acid composition of Bombyx mori silk fibroin and of Tussah silk fibroin, compares the amino acid compositions of the silk from two silkworm species.

US Patent Application Publication. Pub. No. US 2008/0004421 A1: Chenault, H., et al., Tissue adhesives with modified elasticity discloses an adhesive hydrogel useful as a medical tissue adhesive for example to assist wound closure can be made by preparing a chain extended, multi-arm polyether amine (such as an 8 arm PEG amine) cross linked (using for example PEG 4000 dimesylate) to an oxidized polysaccharide (such as dextran), by mixing the cross linked molecule in a syringe at the point of injection or administration with a hydrogel such as a solution of dextran dialdehyde.

US Patent Application Publication. Pub. No. US 2010/0016886 A1: Lu, H., High swell, long lived hydrogel sealant; discusses reacting a multi-arm amine (i.e. an 9 arm polyethelene glycol (PEG) with an oxidized (i.e. to introduce aldehyde groups) polysaccharide (such as hyaluronic acid), useful for tissue augmentation or a tissue adhesive/sealant.

U.S. Pat. No. 6,903,199 to Moon. T., et al., Crosslinked amide derivatives of hyaluronic acid and manufacturing method thereof discusses cross linking hyaluronic acid with a chitosan or with a deacetylated hyaluronic acid with reactive amide groups, using (for example) EDC or NHS.

International Patent Application WO/2010/123945, Altman, G., et al., Silk fibroin hydrogels and uses thereof discusses silk hydrogels made by, for example, digesting degummed silk hydrogels made by, for example, digesting degummed Bombyx mori silk at 60° C. for 4 hours in 9.3M lithium bromide to thereby obtain a 20% silk solution, an 8% silk solution of which was induced to gel using 23RGD and/or ethanol, which can be present in a hyaluronic acid carrier. Altman also discusses possible use as a dermal filler and to promote wound closure, and (in paragraph [0210]) a silk hydrogel coating on a silk mesh. Altman also discusses silk cross linked to hyaluronic acid (see paragraphs [213] to [220], using various cross linkers.

International Patent Application. Pub. No. WO/2008/008857: Prestwich, G., et al., Tholated macromolecules and methods for making and using thereof discloses a thioethyl ether substituted hyaluronic acid made by oxidating coupling useful, for example, in arthritis treatment.

International Patent Application. Pub. No. WO/2008/008859: Prestwich, G., et al., Macromolecules modified with electrophilic groups and methods of making and using thereof discloses a haloacetate derivative hyaluronic acid reacted with thiol modified hyaluronic acid to make a hydrogel, with various medical uses.

Biomacromolecules, 2010, 11 (9), 2230-2237: Serban, M., et. Al., Modular elastic patches: mechanical and biological effects discusses how to make an elastic patch by cross linking elastin, hyaluronic acid and silk, by adding an aminated hyaluronic acid (made using EDC) with a 20% silk solution and elastin, in PBS with BS3 (bissulfosuccinimidyl suberate, as cross linker) at 37° C. for 12 hours.

Biomaterials, 2008, 29(10), 1388-1399: Serban, M., et al., Synthesis, characterization and chondroprotective properties of a hyaluronan thioethyl ether derivative discusses a viscous 2-thioethyl ether hyaluronic acid derivative solution useful for viscosupplementation in arthritis treatment. The abstract mentions that a prior hyaluronic acid with multiple thio groups can be used for adhesion prevention.

Methods, 2008, 45, 93-98: Serban, M., et al., Modular extracellular matrices: solutions to the puzzle discusses cross linked thio modified hyaluronic acid hydrogel useful as a semi synthetic extracellular matrix for cell culture.

Biomacromolecules, 2007, 8(9), 2821-2828: Serban, M., et al., Synthesis of hyaluronan haloacetates and biology of novel cross linker free synthetic extracellular matrix hydrogels discusses cross linking haloacetate substituted hyaluronic acids reacted with a thiol substituted hyaluronic acid to make a hydrogel useful for cell culture or adhesion prevention or medical device coating.

Journal of Materials Chemistry, 2009, 19, 6443-6450: Murphy A., et al., Biomedical applications of chemically modified silk fibroin is a review of methods to make silk conjugates, including silk conjugated to oligosaccharides, modified silk and medical uses.

Biomacromolecules, 2004, 5, 751-757: Sohn, S., et al., Phase behavior and hydration of silk fibroin discusses a study of Bombyx mori silk in vitro using osmotic stress, determining that silk I (α-silk) but not silk II (β-sheet, spun silk fiber) is hydrated.

U.S. Pat. No. 8,071,722 to Kaplan, D., et al., Silk Biomaterials and methods of use thereof discloses silk films, use of 9-12 m LiBr to dissolve extracted silk, adding hyaluronic acid to a silk solution to make fibers from the composition. See also eg the Kaplan patents and application U.S. Pat. Nos. 7,674,882; 8,178,656; 2010 055438, and; 2011 223153.

US patent application 2011 071239 by Kaplan, D., et al., PH induced silk gels and uses thereof discloses methods for making silk fibroin gel from silk fibroin solution, useful to coat a medical device (see paragraph [0012]), as an injectable gel to fill a tissue void, making an adhesive silk gel (with or without a hyaluronic acid), adhering the adhesive silk gel to a subject for example for use as a wound bioadhesive, a multi-layered silk gel.

US patent application 2009 0202614 by Kaplan, D., et al., Methods for stepwise deposition of silk fibroin coatings discusses layered silk coatings, silk films made using silk fibroin solutions which can include a hyaluronic acid, useful, for example, as wound healing patches, to coat an implantable medical device.

U.S. Pat. No. 4,818,291 to Iwatsuki M., et al., Silk-fibroin and human-fibrinogen adhesive composition discusses surgical adhesive useful in tissue repair made as a mixture of LiBr dissolved silk and fibrinogen.

Implantable, knitted silk fabrics for surgical use are known. See eg US patent applications 2004/0224406 and 2012/0029537. Post operative adhesions are a common occurrence after surgery and are undesirable. For example postoperative intra-abdominal and pelvic adhesions are the leading cause of infertility, chronic pelvic pain, and intestinal obstruction. Adhesions form as a result of the body's natural healing response and imply migration of fibroblasts to the trauma/wound site, cell proliferation, de novo extracellular matrix secretion and wound closing through adhesion formations. Post-operative adhesions can occur at the tissue-tissue interface (i.e. peritendinous tissue adhesion involves adhesion between the repaired tendon and the surrounding tissue) or at a tissue-biomaterial interface, in cases where a biomaterial (i.e. a supporting scaffold) is used to reinforce the mechanical properties of the repaired tissue. For example in hernia repair where a biomaterial mesh is used to reinforce the reconstructed abdominal wall, adhesions commonly form between the mesh and underlying bowel tissue.

Thus there is a need for a biomaterial mesh that can decrease or eliminate formation of post-operative adhesions.

### SUMMARY

The present invention meets these needs and provides silk based medical devices that can reduce or prevent post-operative tissue to tissue or tissue to scaffold adhesion formation. Important to the invention was discovery of a biocompatible material that: by its very nature does not promote cell attachment; provides a smooth surface that further hinders cell attachment; eliminate the introduction of foreign chemical agents; exploit silk's intrinsic physical cross linking capacity via hydrogen-bond mediated beta-sheet formation; and; provides robust, pliable, and user friendly medical device.

The present invention also includes an entirely silk based self adherent medical devices. This device is: biocompatible and can stick (adhere) to a physiological surface (such as skin or other tissue surface); provides a smooth surface that can prevent cell adherence and/or tissue abrasions; circumvent the introduction of any external agents or chemicals; makes use of silk's intrinsic physical crosslinking capacity via hydrogen-bond mediated beta-sheet formation; and (e) robust, pliable, cost-efficient and a user friendly medical device.

### DRAWINGS

Aspect of the present invention are illustrated by the following drawings.

FIG. 1 illustrates the procedure for casting a silk form from a silk solution to thereby make a water resistant silk film. The middle drawing in FIG. 1 shows the silk solution being dispensed from a pipette. “EtOH” in FIG. 1 means application of ethanol to the silk film.

FIG. 2 illustrates the procedure for making a multi laminate medical device using the water resistant silk film made by the FIG. 1 process. In FIG. 2 the water resistant silk film is shown fused onto a knitted silk mesh (SeriScaffold).

FIG. 3 is a graph obtained by use of FTIR showing on the x axis the absorbance wavelength (nm) and on the y axis the absorbance (arbitrary units or AU) confirming beta sheet induction through silk film treatment with the ethanol solution.

FIG. 4 shows on the left hand side of FIG. 4 a side view photograph and on the right hand side of FIG. 4 a top view photograph of the water resistant silk film made by the process of FIG. 1.

FIG. 5 is a pictorial representation of how the silk film made by the process of FIG. 1 can be used to wrapped around a portion of a tendon so as to isolate the tendon from adjacent tissues.

FIG. 6 shows on the left hand side of FIG. 6 a bottom view photograph (the “smooth side”) of a multi laminate medical device comprising a water resistant silk film fused to the bottom side of a knitted silk fabric. The right hand side of FIG. 6 is a top view photograph (the “rough side”) of the multi laminate silk device.

FIG. 7 is a pictorial representation showing in the top portion of FIG. 7 knit characteristics of the knitted silk fabric used (SeriScaffold), and in the bottom portion of FIG. 7 . how the film has fused into the silk fabric.

FIG. 8 is a pictorial representation of the use of the fused silk-film mesh medical device for post-operative adhesion prevention in an abdominal wall repair model.

FIG. 9 is an illustration of the casting process of a double layered self-adherent silk film.

FIG. 10 is a graph obtained by use of FTIR showing on the x axis the absorbance wavelength (nm) and on the y axis the absorbance (AU) confirming beta sheet induction through silk film treatment with the ethanol solution.

FIG. 11 present two photographs of a multi laminate (two layers of silk film) medical device, showing in the left hand side photograph adherence to the top of a Petri dish and in the right hand side photograph adherence to a moistened nitrile surgical glove.

FIG. 12 is a pictorial illustration of the ilk film adherence mechanism to wet or moist surfaces. The hydrophilicity of the contact surface probably triggers silk fibroin structural rearrangements that lead to the reorientation of the hydrophilic and hydrophobic regions of the protein to promote the most energetically favorable interactions.

### DESCRIPTION

The present invention is based on the discovery of laminate silk medical devices that can be implanted to separate adjoining tissues, provide soft tissue support and/or reduce formation of adhesion.

The silk films and the silk fabrics set forth herein can be made from silkworm cocoons substantially depleted of sericin. A preferred source of raw silk is from the silkworm B. mori. Other sources of silk include other strains of Bombycidae including Antheraea pernyi, Antheraea yamamai, Antheraea mylitta, Antheraea assama, and Philosamia cynthia ricini, as well as silk producing members of the families Saturnidae, Thaumetopoeidae, and silk-producing members of the order Araneae. Suitable silk can also be obtained from other spider, caterpillar, or recombinant sources. Methods for performing sericin extraction have been described in pending U.S. patent application Ser. No. 10/008,924, U.S. Publication No. 2003/0100108, Matrix for the production of tissue engineered ligaments, tendons and other tissue.

Extractants such as urea solution, hot water, enzyme solutions including papain among others which are known in the art to remove sericin from fibroin would also be acceptable for generation of the silk. Mechanical methods may also be used for the removal of sericin from silk fibroin. This includes but is not limited to ultrasound, abrasive scrubbing and fluid flow. The rinse post-extraction is conducted preferably with vigorous agitation to remove substantially any ionic contaminants, soluble, and in soluble debris present on the silk as monitored through microscopy and solution electrochemical measurements. A criterion is that the extractant predictably and repeatably remove the sericin coat of the source silk without significantly compromising the molecular structure of the fibroin. For example, an extraction may be evaluated for sericin removal via mass loss, amino acid content analysis, and scanning electron microscopy. Fibroin degradation may in turn be monitored by FTIR analysis, standard protein gel electrophoresis and scanning electron microscopy.

In certain cases, the silk utilized for making the composition has been substantially depleted of its native sericin content (i.e., ≦4% (w/w) residual sericin in the final extracted silk). Alternatively, higher concentrations of residual sericin may be left on the silk following extraction or the extraction step may be omitted. In preferred aspects of this embodiment, the sericin-depleted silk fibroin has, e.g. about 0% to about 4% (w/w) residual sericin. In the most preferred aspects of this embodiment, the sericin-depleted silk fibroin has, e.g. about 1% to 3% (w/w) residual sericin.

In certain cases, the silk utilized for generation of a medical device within the scope of the present invention is entirely free of its native sericin content. As used herein, the term “entirely free (i.e. “consisting of” terminology) means that within the detection range of the instrument or process being used, the substance cannot be detected or its presence cannot be confirmed.

The water soluble or dissolved silk can be prepared by a 4 hour digestion at 60° C. of pure silk fibroin at a concentration of 200 g/L in a 9.3 M aqueous solution of lithium bromide to a silk concentration of 20% (w/v). This process may be conducted by other means provided that they deliver a similar degree of dissociation to that provided by a 4 hour digestion at 60° C. of pure silk fibroin at a concentration of 200 g/L in a 9.3 M aqueous solution of lithium bromide. The primary goal of this is to create uniformly and repeatably dissociated silk fibroin molecules to ensure similar fibroin solution properties and, subsequently, device properties. Less substantially dissociated silk solution may have altered gelation kinetics resulting in differing final gel properties. The degree of dissociation may be indicated by Fourier-transform Infrared Spectroscopy (FTIR) or x-ray diffraction (XRD) and other modalities that quantitatively and qualitatively measure protein structure. Additionally, one may confirm that heavy and light chain domains of the silk fibroin dimer have remained intact following silk processing and dissolution. This may be achieved by methods such as standard protein sodium-dodecyl-sulfate polyacrylamide gel electrophoresis (SDS-PAGE) which assess molecular weight of the independent silk fibroin domains.

System parameters which may be modified in the initial dissolution of silk include but are not limited to solvent type, silk concentration, temperature, pressure, and addition of mechanical disruptive forces. Solvent types other than aqueous lithium bromide may include but are not limited to aqueous solutions, alcohol solutions, 1,1,1,3,3,3-hexafluoro-2-propanol, and hexafluoroacetone, 1-butyl-3-methylimidazolium. These solvents may be further enhanced by addition of urea or ionic species including lithium bromide, calcium chloride, lithium thiocyanate, zinc chloride, magnesium salts, sodium thiocyanate, and other lithium and calcium halides would be useful for such an application. These solvents may also be modified through adjustment of pH either by addition of acidic of basic compounds.

The medical devices disclosed herein is preferably biodegradable, bioerodible, and/or bioresorbable. In an embodiment the medical device (as a silk film) does not entirely or substantially biodegrade between about 10 days to about 120 days after implantation. In an embodiment the medical device (as a laminate silk device with both a silk film and a silk fabric) does not entirely or substantially biodegrade between about 300 days to about 600 days after implantation.

Aspects of the present specification provide, in part, a silk film having a transparency and/or translucency. Transparency (also called pellucidity or diaphaneity) is the physical property of allowing light to pass through a material, whereas translucency (also called translucence or translucidity) only allows light to pass through diffusely. The opposite property is opacity. Transparent materials are clear, while translucent ones cannot be seen through clearly. The silk films disclosed herein may, or may not, exhibit optical properties such as transparency and translucency. In certain cases, e.g., superficial line filling, it would be an advantage to have an opaque silk film. In other cases such as development of a lens or a “humor” for filling the eye, it would be an advantage to have a translucent silk film. These properties could be modified by affecting the structural distribution of the silk film. Factors used to control a hydrogel's optical properties include, without limitation, silk fibroin concentration, gel crystallinity, and silk homogeneity.

When light encounters a material, it can interact with it in several different ways. These interactions depend on the nature of the light (its wavelength, frequency, energy, etc.) and the nature of the material. Light waves interact with an object by some combination of reflection, and transmittance with refraction. As such, an optically transparent material allows much of the light that falls on it to be transmitted, with little light being reflected. Materials which do not allow the transmission of light are called optically opaque or simply opaque.

In an embodiment, a silk film is optically transparent. In aspects of this embodiment, a silk film transmits, e.g., between about 75% to about 100% of the light. In some preferred aspects of this embodiment, a silk film transmits, e.g., between about 80% to about 90% of the light. In the most preferred aspects of this embodiment, a silk film transmits, e.g., between about 85% to about 90% of the light.

Aspects of the present specification provide, in part, a medical device comprising a hyaluronan. As used herein, the term “hyaluronic acid” is synonymous with “HA”, “hyaluronic acid”, and “hyaluronate” refers to an anionic, non-sulfated glycosaminoglycan polymer comprising disaccharide units, which themselves include D-glucuronic acid and D-N-acetylglucosamine monomers, linked together via alternating β-1,4 and β-1,3 glycosidic bonds and pharmaceutically acceptable salts thereof. Hyaluronan can be purified from animal and non-animal sources. Polymers of hyaluronan can range in size from about 5,000 Da to about 20,000,000 Da. Any hyaluronan is useful in the compositions disclosed herein with the proviso that the hyaluronan improves a condition of the skin, such as, e.g., hydration or elasticity. Non-limiting examples of pharmaceutically acceptable salts of hyaluronan include sodium hyaluronan, potassium hyaluronan, magnesium hyaluronan, calcium hyaluronan, and combinations thereof.

Aspects of the present specification provide, in part, a composition comprising a crosslinked matrix polymer. As used herein, the term “crosslinked” refers to the intermolecular bonds joining the individual polymer molecules, or monomer chains, into a more stable structure like a gel. As such, a crosslinked matrix polymer has at least one intermolecular bond joining at least one individual polymer molecule to another one. Matrix polymers disclosed herein may be crosslinked using dialdehydes and disufides crosslinking agents including, without limitation, multifunctional PEG-based cross linking agents, divinyl sulfones, diglycidyl ethers, and bis-epoxides. Non-limiting examples of hyaluronan crosslinking agents include divinyl sulfone (DVS), 1,4-butanediol diglycidyl ether (BDDE), 1,2-bis(2,3-epoxypropoxy)ethylene (EGDGE), 1,2,7,8-diepoxyoctane (DEO), biscarbodiimide (BCDI), pentaerythritol tetraglycidyl ether (PETGE), adipic dihydrazide (ADH), bis(sulfosuccinimidyl)suberate (BS), hexamethylenediamine (NMDA), 1-(2,3-epoxypropyl)-2,3-epoxycyclohexane, or combinations thereof.

Aspects of the present specification provide, in part, a composition comprising a crosslinked matrix polymer having a degree of crosslinking. As used herein, the term “degree of crosslinking” refers to the percentage of matrix polymer monomeric units that are bound to a cross-linking agent, such as, e.g., the disaccharide monomer units of hyaluronan. Thus, a composition that that has a crosslinked matrix polymer with a 4% degree of crosslinking means that on average there are four crosslinking molecules for every 100 monomeric units. Every other parameter being equal, the greater the degree of crosslinking, the harder the gel becomes. Non-limiting examples of a degree of crosslinking include about 1% to about 15%.

In an embodiment, a composition comprises an uncrosslinked hyaluronan where the uncrosslinked hyaluronan comprises a combination of both high molecular weight hyaluronan and low molecular weight hyaluronan in a ratio of about 20:1, about 15:1, about 10:1, about 5:1, about 1:1, about 1:5 about 1:10, about 1:15, or about 1:20.

In another embodiment, a composition comprises an uncrosslinked hyaluronan where the uncrosslinked hyaluronan comprises a combination of both high molecular weight hyaluronan and low molecular weight hyaluronan, in various ratios. As used herein, the term “high molecular weight hyaluronan” refers to a hyaluronan polymer that has a molecular weight of 1,000,000 Da or greater. Non-limiting examples of a high molecular weight hyaluronan include a hyaluronan of about 1,500,000 Da, a hyaluronan of about 2,000,000 Da, a hyaluronan of about 2,500,000 Da, a hyaluronan of about 3,000,000 Da, a hyaluronan of about 3,500,000 Da, a hyaluronan of about 4,000,000 Da, a hyaluronan of about 4,500,000 Da, and a hyaluronan of about 5,000,000 Da. As used herein, the term “low molecular weight hyaluronan” refers to a hyaluronan polymer that has a molecular weight of less than 1,000,000 Da. Non-limiting examples of a low molecular weight hyaluronan include a hyaluronan of about 200,000 Da, a hyaluronan of about 300,000 Da, a hyaluronan of about 400,000 Da, a hyaluronan of about 500,000 Da, a hyaluronan of about 600,000 Da, a hyaluronan of about 700,000 Da, a hyaluronan of about 800,000 Da, and a hyaluronan of about 900,000 Da.

In other aspects of this embodiment, a composition comprises a crosslinked hyaluronan where the crosslinked hyaluronan has a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In yet other aspects of this embodiment, a composition comprises a crosslinked hyaluronan where the crosslinked hyaluronan has a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In still other aspects of this embodiment, a composition comprises a crosslinked hyaluronan where the crosslinked hyaluronan has a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da.

In other aspects of this embodiment, a composition comprises an uncrosslinked hyaluronan where the uncrosslinked hyaluronan has a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In yet other aspects of this embodiment, a composition comprises an uncrosslinked hyaluronan where the uncrosslinked hyaluronan has a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In still other aspects of this embodiment, a composition comprises an uncrosslinked hyaluronan where the uncrosslinked hyaluronan has a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da. In further aspects, a composition comprises an uncrosslinked hyaluronan where the uncrosslinked hyaluronan has a mean molecular weight of, e.g., greater than 2,000,000 Da and less than about 3,000,000 Da, greater than 2,000,000 Da and less than about 3,500,000 Da, greater than 2,000,000 Da and less than about 4,000,000 Da, greater than 2,000,000 Da and less than about 4,500,000 Da, greater than 2,000,000 Da and less than about 5,000,000 Da.

### EXAMPLES

The following examples illustrate embodiments of the present invention.

### Example 1

### Preparation of a Silk Based Biomaterial Useful as an Adhesion Barrier

The materials used in this Example 1 to make a silk based biomaterial useful as an adhesion barrier included: an aqueous silk fibroin solution (7-12% w/v concentration of silk); sterile 60-mm Petri dishes (used as casting molds); ethanol solution 90% v/v, and; a knitted silk fabric (the particular knitted silk fabric used was SeriScaffold® surgical scaffold available from Allergan, Inc., Irvine, Calif. SeriScaffold is made as and has the properties set forth in U.S. patent application Ser. No. 13/715,872.

To obtain a solution of water-soluble silk fibroin, Bombyx Mori silk cocoons were obtained and first soaked in a warm basic solution to remove the immunogenic protein sericin naturally present on the silkworm silk. The sericin depleted silk was then digested by dissolving the silk in 9.3M LiBr and then dialyzed in an aqueous solution of denaturated and dissolved state. The amino acid composition of Bombyx Mori silk fibroin shows a low amount of aspartic acid/glutamic acid (carboxylic groups), even lower amount of lysine (amine groups) and a high amount of serine (hydroxyl groups). Silk beta-sheet formation can be induced with accelerants (pH, temperature, vortexing, sonication, ethanol treatment, etc.

A first device was made as follows. Silk fibroin solution (1 ml) was cast on the bottom of an inverted 60 mm Petri dish and allowed to dry between 2-12 hours (see FIG. 1). The dried films were then immersed for two 2 hours in the ethanol solution to induce beta-sheet formation in the silk.

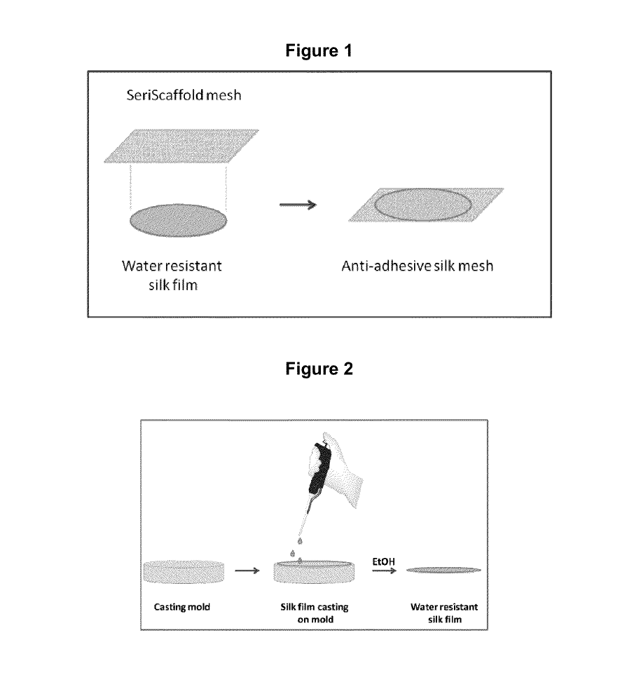

A second device was made as follows. Silk fibroin films cast as described above were allowed to dry for 50 minutes in a laminar flow hood then, prior to complete drying of the surface, were overlayed with precut SeriScaffold meshes (4×5 cm) (see FIG. 2). The film was allowed to fuse with the mesh for 2-12 hours, then the construct was immersed in ethanol solution for 2 hours to induce physical crosslinking via beta sheets.

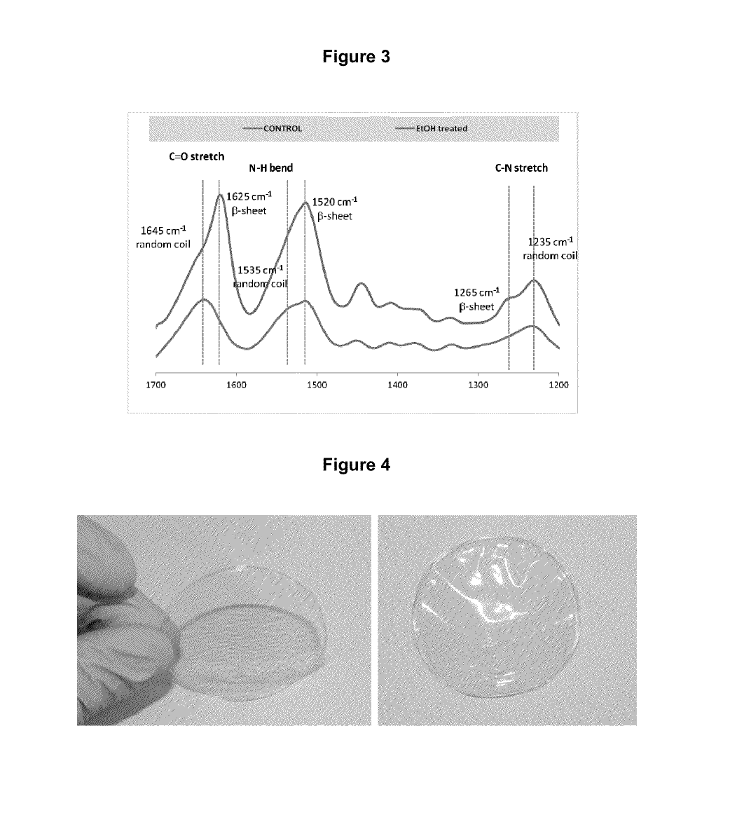

For both devices made in this Example 1, the ability of silk to become water resistant by physical crosslinking of the silk molecules was made use of. Through this cross linking process, the silk fibroin protein underwent structural rearrangements to a beta-sheet rich conformation. Temperature, pH, ionic strength and treatment with polar agents such as alcohols are all factors known to induce such structural transitions. For the two devices made in this Example 1, beta sheet formation was induced via ethanol treatment (see FIG. 3).

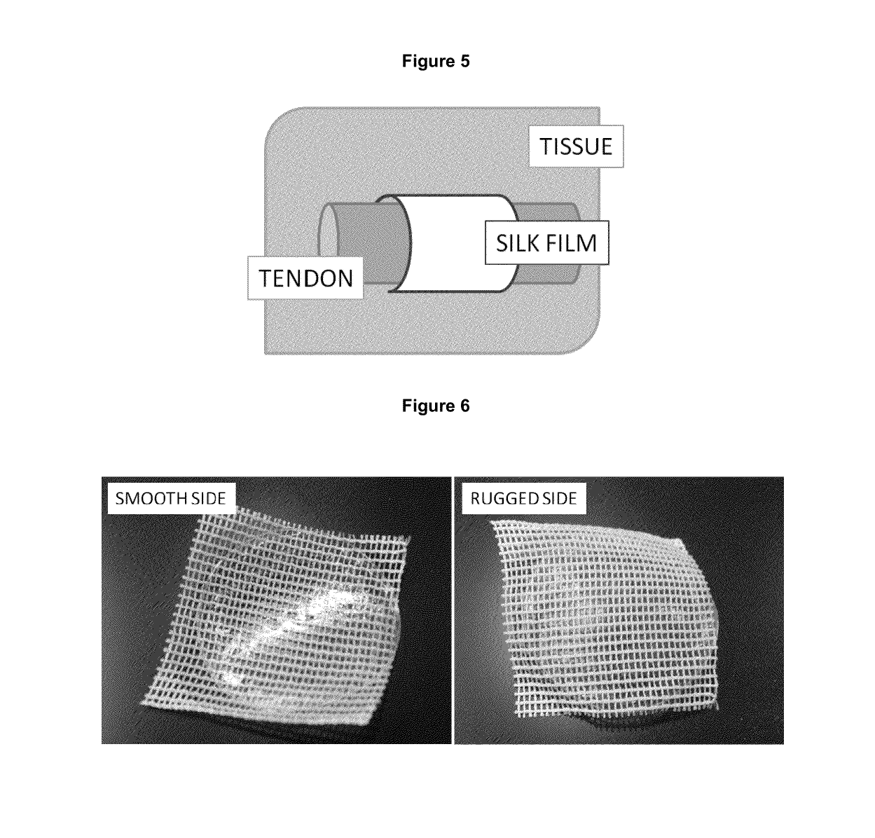

The first device was a monolayer of transparent water-resistant silk film, as shown by FIG. 4. The thickness of the film was controllable and depended on the silk fibroin solution concentration and the casting area. We found that an 8% w/v silk fibroin solution cast on a 4.6 cm diameter mold would yield a 50 μm tick film. The film was pliable, moldable, stretchable, with good mechanical integrity (average maximum load of 8.8±1.9 N for a 50 μm thick film versus an average maximum load of 71.7±1.0 N of SeriScaffold) and can be used to wrap the target tissue (i.e. tendon) to isolate it from the surrounding tissues to with it may non-specifically adhere (FIG. 5). Additionally, the first device can be used in conjunction with other devices (meshes, sheets). Moreover, the transparency of device 1 is a convenient feature as it allows the user to correctly evaluate the positioning of the device 1 film on the tissue.

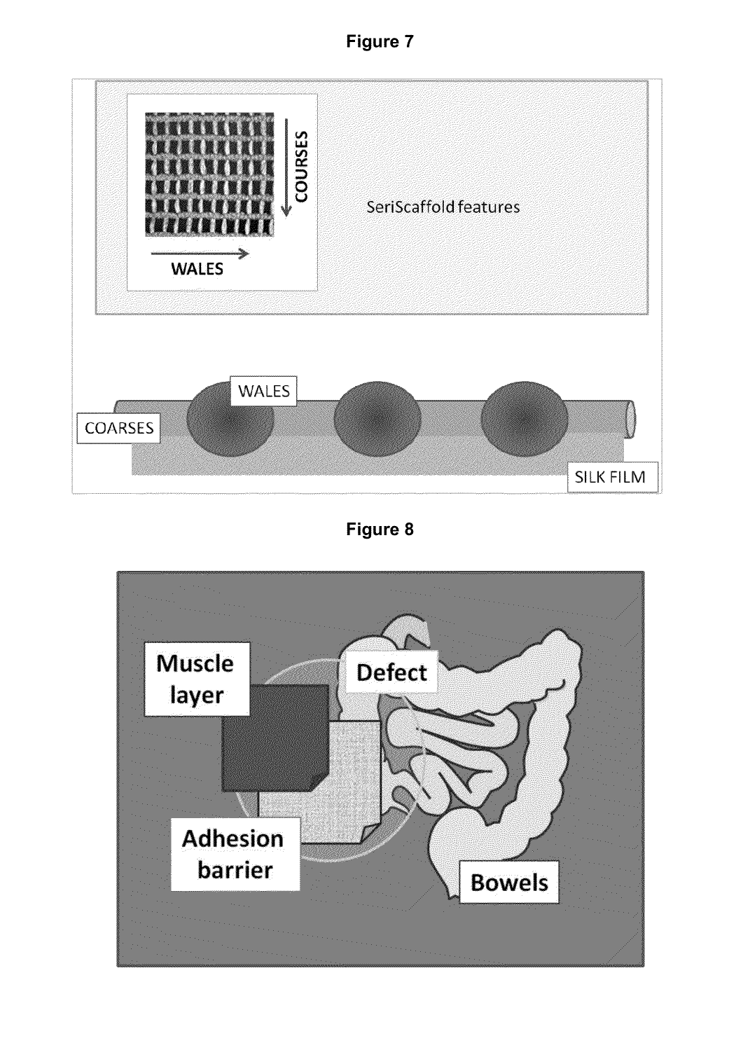

The second device made in this Example 1 consisted of a single layer silk film fused with the SeriScaffold (see FIG. 6). The fusion of the silk with the mesh was driven by the partial encasing of the mesh filaments by the silk solution prior its complete drying (FIG. 7). After complete drying of the film the construct was treated with ethanol solution to render it water insoluble via beta-sheet formation. The key features of this second device were: (a)—its smooth surface on one side and (b)—the rugged surface provided by the mesh pores on the other side. In the case of the abdominal wall repair for example, the smooth side is intended to contact the bowel and prevent adhesion formations, while the rugged surface will face the abdominal wall and will integrate well with the surrounding tissue by promoting cells to adhere to its groove (FIG. 8).

Both device 1 and device 2 have the advantage of being both entirely silk fibroin based. The sterility of both these devices can be ensured either by using autoclaved silk fibroin solution for film casting (and fusing them with sterile meshed for device 2) or via ethylene oxide sterilization. Moreover, both devices are compatible to be used with a variety of other mesh medical such as Vicryl and Mersilene. These devices: (a)—are biocompatible and do not intrinsically sustain cell attachment as previously established by large bodies of scientific literature; (b)—provide a smooth surface that further hinders cell attachment; (c)—do not contain any “foreign” chemical agents; (d)—are physically crosslinked through intra- and inter-molecular beta-sheets; and (e)—are robust, drapable and easy to handle.

### Example 2

### Self Adherent Silk Based Biomaterials

The materials used in this Example 2 included: an aqueous silk fibroin solution (7-12% w/v) made by the same methods set forth in Example 2; sterile 60-mm Petri dishes (used as casting molds), and; an ethanol solution 90% v/v.

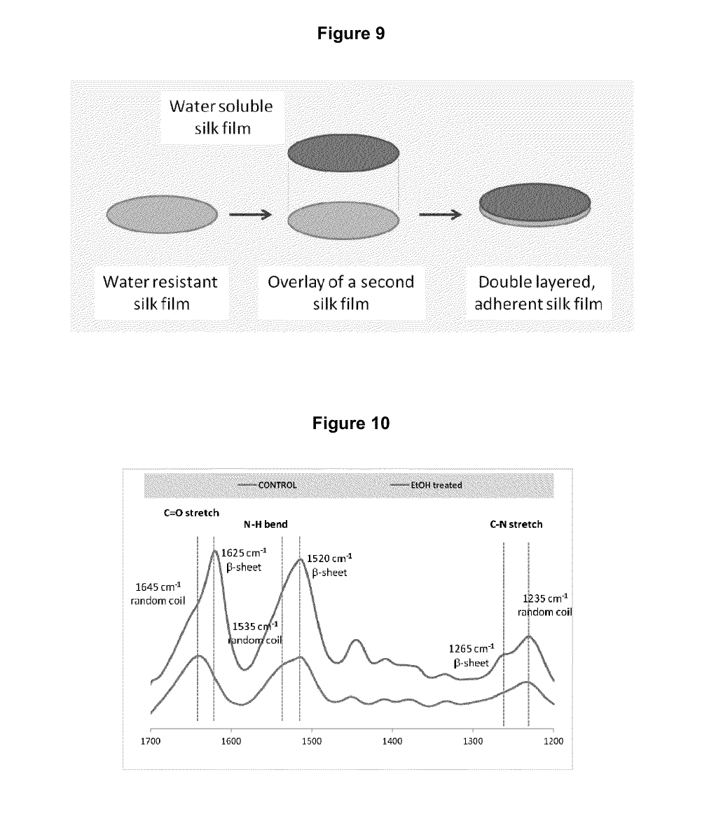

Silk fibroin solution (8% w/v, 1 ml) was cast on the bottom of two inverted 60 mm Petri dish and allowed to dry between 2-12 hours. Half of the films were then immersed for 2 hours in ethanol solution to induce beta-sheet formation. Subsequently, the ethanol treated films were rinsed with deionized water and repositioned on the molds. The remaining films (non-treated, water soluble silk films) were then deposited on top of the wet ethanol treated films and the double layered films was allowed to air dry for 2-12 hours (FIG. 9). Alternatively, a second layer of silk fibroin solution was deposited on top of the ethanol treated films, then allowed to dry, to yield the double layered self-adherent films.

This Example 2 also made use of silk's natural ability to become water resistant via physical crosslinking. Through this process, the silk fibroin protein undergoes structural rearrangements to a beta-sheet rich conformation. Temperature, pH, ionic strength and treatment with polar agents such as alcohols are all factors known to induce such structural transitions. For the device made in this Example 2, beta sheet formation was induced via ethanol treatment (FIG. 10).

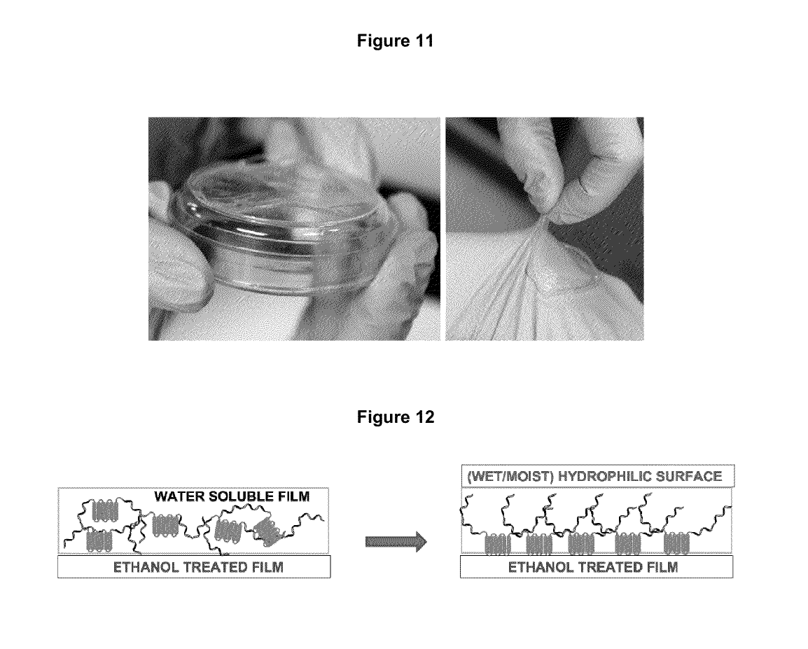

The devices made were smooth, double layered, self-adherent silk film consisting of a waterproof, physically crosslinked side and a water soluble, adherent side. The adhesiveness of the water soluble silk film is responsible for the cohesiveness of the double layered constructs as it intimately blended with the surface of the ethanol treated film. The dried device can be easily handled with dried gloves or hands. When applied to a wet or moist surface, the water soluble side of construct rehydrates and tightly adheres to the contact surface (FIG. 11). The ethanol treated side then provides a beta sheet rich, waterproof barrier.

The film adherence mechanism probably implies structural rearrangements of the silk fibroin in which the hydrophilic regions of the protein get oriented toward and interact with the hydrophilic regions of the contact surface and analogously, the hydrophobic regions of the protein re-orient toward and interact with the hydrophobic, beta sheet rich interface of the ethanol treated silk film (FIG. 12).

The device can be used for example in: (a)—hemostasis (by attaching it to bleeding blood vessels); (b)—wound dressing (by attaching it to superficial wounds); (c)—burn dressings (by substituting skin grafts); (d)—small defect repair patch (by patching small defects such a tympanic membrane holes); (e)—tissue enforcing/supporting patch (by wrapping it against weakened tissues, i.e. cervix to prevent pre-term deliveries); or (f)—post-operative adhesion barrier (by attaching it to the affected tissue with the “sticky” side, then the waterproof side would serve as a barrier to attachment to surrounding tissues). The versatility of this device is further highlighted by its transparency—which would enhance the ability to control the exact placement of the device; ease of sterilization—since it can be sterilely manufactured from autoclaved silk fibroin solution; control over the thickness and mechanical strength —since these parameters are dictated by the concentration of the silk solution used and the cast mold area; prolonged stability and cost effective manufacturing process.

### Example 3

### Use of Silk Medical Device in Abdominal Surgery

Briefly, a hernia is a bulge of intestine, another organ, or fat through the muscles of the abdomen, where tissue structure and function is lost at the load-bearing muscle, tendon and fascial layer. Thus, a hernia can occur when there is weakness in the muscle wall that allows part of an internal organ to push through. The silk medical device within the scope of the present invention can be used to assist in the repair of an inguinal (inner groin), incisional (resulting from an incision), femoral (outer groin), umbilical (belly button), or hiatal (upper stomach) hernia, using either an open or laproscopic technique. A ventral hernia is a type of abdominal hernia—it can develop as a defect at birth, resulting from incomplete closure of part of the abdominal wall, or develop where an incision was made during an abdominal surgery, occurring when the incision doesn't heal properly.

A silk medical device within the scope of the present invention can be used in both open and laparoscopic procedures to assist in the repair of a ventral hernia as follows: the patient lies on the operating table, either flat on the back or on the side, depending on the location of the hernia. General anesthesia is usually given, though some patients can have local or regional anesthesia, depending on the location of the hernia and complexity of the repair. A catheter is inserted into the bladder to remove urine and decompress the bladder. If the hernia is near the stomach, a gastric (nose or mouth to stomach) tube can be inserted to decompress the stomach. In an open procedure, an incision is made just large enough to remove fat and scar tissue from the abdominal wall near the hernia. The outside edges of the weakened hernial area are defined and excess tissue removed from within the area. The silk medical device s then applied so that it overlaps the weakened area by several inches (centimeters) in all directions. Non-absorbable sutures are placed into the full thickness of the abdominal wall. The sutures are tied down and knotted.

In the less-invasive laparoscopic procedure, two or three small incisions are made to access the hernia site—the laparoscope is inserted in one incision and surgical instruments in the others to remove tissue and place the silk medical device in the same fashion as in an open procedure. Significantly less abdominal wall tissue is removed in laparoscopic repair. The surgeon views the entire procedure on a video monitor to guide the placement and suturing of the silk medical device.

In closing, it is to be understood that although aspects of the present specification have been described with reference to the various embodiments, one skilled in the art will readily appreciate that the specific examples disclosed are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A laminate, implantable silk medical device comprising:
(a) a first layer comprising a knitted silk fabric, the first later having a top side and a bottom side, and;
(b) a second layer comprising a silk film fused to at least a portion of the bottom side of the first layer, thereby obtaining a laminate, implantable silk medical device.

2. The medical device of claim 1, wherein the silk film is water resistant.

3. The medical device of claim 1, wherein the silk film is fused to the silk fabric by drying the silk film after placing the silk film onto the silk fabric.

4. A process for making a laminate, implantable silk medical device, the process comprising:
(a) knitting a fabric from sericin depleted silk thereby making a first layer having a top side and a bottom side, and;
(b) preparing a silk solution by dissolving silk into to a solvent;
(c) casting a silk film from the silk solution;
(d) treating the silk film so that at least one side of the silk film is water resistant, thereby forming a second layer; and
(e) fusing the second layer to at least a portion of the bottom side of the first layer, thereby obtaining a laminate, implantable silk medical device.

5. A method for providing tissue support and reducing adhesion formation, the method comprising the steps of implanting the device of claim 1.

6. An abdominal surgical method comprising the step of implanting the device of claim 1.

7. A laminate, implantable silk medical device comprising:
(a) a first layer comprising a water resistant, non-adherent silk film, the first layer having a top side and a bottom side, and;
(b) a second layer comprising a water soluble, adherent silk film formed on or placed on the top side of the first layer, thereby obtaining a laminate, implantable silk medical device.
